(19) United States
(12) Patent Application Publication
(10) Pub. No.: US 2024/0315728 A1
(43) Pub. Date: Sep. 26, 2024

(54) NEONATAL PNEUMOTHORAX DECOMPRESSION DEVICE

(71) Applicant: Regents of the University of Minnesota, Minneapolis, MN (US)

(72) Inventors: Jonathan Strutt, Minneapolis, MN (US); Kari Roberts, Minneapolis, MN (US); Clay Ansley, Minneapolis, MN (US); Kyle Johnson, Minneapolis, MN (US); Sam Keller, Minneapolis, MN (US); Christina Letness, Minneapolis, MN (US); Matthew McCollor, Minneapolis, MN (US)

(21) Appl. No.: 18/611,953

(22) Filed: Mar. 21, 2024

Related U.S. Application Data
(60) Provisional application No. 63/453,617, filed on Mar. 21, 2023.
(60) Provisional application No. 63/606,285, filed on Dec. 5, 2023.

(51) Int. Cl. A61B 17/34; A61B 17/00; A61M 1/04

(52) U.S. Cl. CPC A61B 17/3401; A61M 1/04; A61B 2017/00407; A61B 2017/3492; A61M 2205/073; A61M 2240/00

(57) ABSTRACT

A neonatal pneumothorax decompression device is disclosed. The device comprises a needle stabilizer, a pressure indicator, and a fluid evacuation pump. The needle stabilizer is configured to facilitate insertion of the needle into a subject's pleural cavity and to hold a needle at an adjustable length. The pressure indicator is coupled to the proximal end of the needle stabilizer and is configured to visibly display pressure within the subject's pleural cavity. The fluid evacuation pump is configured to be actuated by a user to evacuate air or fluid from the subject's pleural cavity through the needle. The fluid evacuation pump is proximally coupled to the pressure indicator and the needle stabilizer.

### RELATED APPLICATIONS

This application claims priority to U.S. Provisional Application No. 63/453,617, including Appendices A-M, which was filed on Mar. 21, 2023, and U.S. Provisional Application No. 63/606,285, including Appendices A-M, which was filed on Dec. 5, 2023. The entire content of the applications referenced above are hereby incorporated by reference herein.

### FIELD

The present application relates to a pneumothorax decompression device, particularly for use in neonates.

### BACKGROUND

Pneumothorax is a life-threatening condition in which air escapes the lungs causing the chest wall to expand and compress the lungs. Without proper treatment pneumothorax can be fatal in children and adults. Despite the severity of this condition, few commercial devices exist for treatment in adults, none of which are applicable to children and newborns.

The onset of pneumothorax is nondiscriminatory and can happen to anyone. Various causes are known for this condition, many of which are age dependent. Despite being triggered by different events, once onset, pneumothorax is nearly identical across all age groups and body types. Pneumothorax, often referred to as “pneumo” for short, describes the inflation of the pleural cavity which in turn compresses the lungs and prevents normal respiration. The pleural cavity is located along the inside of the chest wall and normally maintains negative pressure relative to atmosphere throughout the respiration cycle, which induces the elastic recoil of the lungs. Without a significant pressure difference between the pleural cavity and pulmonary alveoli, the lungs are unable to properly inflate. The alveoli are miniscule cavities in the lungs which promote the transfer of oxygen and carbon dioxide in the body. Millions of alveoli exist in the lungs and when ruptured they begin to leak gas into the pleural cavity rather than back through the circulatory system. If the ruptured alveoli are permitted to continue leaking air the pleural cavity will reach the same pressure as the pulmonary alveoli, thus defining the onset of pneumothorax.

The vital capacity of a person is defined as the maximum volume of air a person can inhale after complete exhale. This value is critical in defining the wellbeing of a patient and even seemingly slight pressure increases in the pleural space can induce a drastic reduction in the vital capacity of the average adult. In a study of 12 adults, an increase in pleural pressure from −5 cm H₂O to −2 cm H₂O resulted in a 33% decrease in the pulmonary vital capacity.¹ This trend may be reflected to a greater extent in newborns based on scaling laws for volume, though data on this relationship is not readily available due to the severity of pneumothorax in neonates or newborns. While healthy, it was found that the pleural pressure in neonates' range anywhere from −2 cm H₂O to −5 cm H₂O.² According to a study of three neonates, all experiencing spontaneous left-side pneumothoraces, the average change in pleural pressure over 10 breaths was 5.7 cm H₂O.³ Despite resting pleural pressures in adults and neonates holding similar values, the change in pleural pressure during pneumothorax is significantly greater in neonates. The total increase in pleural cavity pressure is dependent on the classification of pneumothorax.

The most common form of pneumothorax is Primary Spontaneous Pneumothorax (PSP), which disproportionately affects current and former smokers. In the case of PSP, subpleural bullae, small air sacs which form between the lung tissue and pleural, release gas into the pleural cavity. This type of pneumothorax is rarely life threatening and usually causes minor to mild discomfort in the patient. While often still requiring treatment, the situation is not nearly as time sensitive as other forms of pneumothorax. Secondary Spontaneous Pneumothorax (SSP) is similar to PSP, however, it results from pulmonary disorders which have the potential to create multiple bullae, resulting in a more severe pneumothorax. The most severe form of pneumothorax is known as a Tension Pneumothorax. This occurs when the pleural alveoli rupture and allow the flow of air directly into the pleural cavity during inhalation. This drastically increases the pleural pressure and forces the associated lung to completely collapse. A tension pneumothorax can be brought about spontaneously or via trauma such as stabbings and gunshot wounds. Without immediate evacuation of the pleural cavity, a tension pneumothorax will lead to death.

Due to the underdeveloped nature of the newborn anatomy, tension pneumothorax is the most common form of pneumothorax in neonates. Rather than being onset by trauma, the most common cause for pneumothorax onset in neonates is due to a lack of surfactant production. Surfactant serves the sole purpose of reducing surface tension at the interface between air and liquid within the human body, thus preventing the rupture of alveoli. With the more recent introduction of medication such as CUROSURF®, a synthetic surfactant, the frequency of pneumothorax in neonates has significantly decreased.⁴ Despite new medication, tension pneumothorax is still present among neonates as surfactant medication is a preventative measure which may not be taken if clear need has not been indicated.

While preventative measures may be taken to reduce the risk of pneumothorax, the rate of occurrence is not significant enough to promote the use of such measures under normal circumstances. One study reported that of 48,968 live births, roughly 0.14% developed pneumothorax with a median age of 28.5 h. Of those newborns displaying pneumothorax, nearly all were born with respiratory illness.⁵ This percentage is roughly indicative of the overall market for a neonatal decompression device. Despite the market size for such a device, the side effects of pneumothorax and necessity for rapid treatment warrant the design of a unique device for the specific treatment of neonatal pneumothorax.

Symptoms and side effects of pneumothorax in adults include discomfort, shortness of breath, and in more severe cases dyspnea, categorized as mild labored breathing. If left untreated, a patient can develop a hypotension and the pneumothorax will eventually lead to death. The time sensitivity in adults is much less crucial as the lungs are fully developed and capable of operation at reduced capacity. In neonates and newborns pneumothorax is much more severe as the lungs are not fully developed and thus incapable of operation under less-than-ideal conditions. The effects of pneumothorax in neonates are severe dyspnea and death, thus having a method for rapid treatment is essential to decreasing the mortality rate of neonates.

The current procedure for neonatal pneumothorax decompression is relatively universal, with minor changes based on the practitioner. In all cases, the device used is made up entirely of off the shelf medical components, adding up to an average cost of no more than $2.00. The device most commonly used consists of a 60 cc syringe, a 3-way stopcock, medical tubing, and a 23 G-25 G hypodermic needle. Slight variations of this device replace the hypodermic needle with a butterfly needle or the syringe with a saline solution, allowing for automatic decompression over time. The needle is connected to the stopcock via medical tubing which is then connected to the syringe.

The presence of a pneumothorax is often confirmed via the use of x-ray imaging as indicated above to avoid unnecessary treatment and clearly indicate the specific cavity region. The x-ray process may take up to 30 minutes under normal conditions and once confirmed, treatment lasts only a few minutes, adhering to the following steps:
1. The second intercostal space is located just above the third rib and cleaned.
2. The patient is put on nearly 100% oxygen to facilitate circulation.
3. The needle is inserted until the syringe can be moved, indicating the cavity was reached.
4. The needle is held in place by one practitioner while another actuates the syringe.
5. Air is pulled from the cavity and the stopcock valve is opened to expel the air.
6. The previous step is repeated until relatively normal respiration is observed.

If the saline solution is used in lieu of a syringe, the procedure can be completed by a single practitioner, however, the rate at which air is evacuated can no longer be controlled. As time is essential to the treatment of pneumothorax in neonates, many practitioners prefer the use of a syringe over a saline solution. It is also common practice to keep a fully assembled device bedside in the Neonatal Intensive Care Unit (NICU) for neonates who present significant risk.

Several key features make the process significantly more sensitive in neonates when compared to adults. The lack of surfactant along the surface of the lungs proposes serious risk of puncturing the lungs or inducing additional air flow into the pleural cavity. The pleural space is also significantly smaller in neonates than adults, leading to the possibility of failed needle insertion. Additionally, the skin of neonates is extremely delicate and prone to damage under even slight pressure. These factors combine to create a unique procedure where extra care must be taken during needle insertion and evacuation. Training for neonatal pneumothorax decompression takes several unorthodox approaches to mimicking the average chest wall of a neonate. Several neonatologists report the use of objects such as Styrofoam cups and bell peppers in training for this procedure. The difficulty associated with this procedure is directly associated with the method of indicating that the needle has reached the pleural cavity. The current method of indication involves feeling the needle enter the cavity and/or the slight movement of the syringe post cavity insertion. Despite the complexity of indicating the cavity has been reached, many experienced practitioners report minimal errors using this method.

In adults the process is more straightforward due to increased size of the pleural cavity and the fully developed nature of the human anatomy. The needle is commonly inserted in the second intercostal space as well, however, the inclusion of a syringe is not needed. The pressure build-up in adults is significant enough to warrant a single one-way valve mounted on the end of the syringe. Upon reaching the cavity, air will flow from the high-pressure region to the low-pressure region to equilibrate the pleural cavity with atmospheric pressure. There are several devices which exist for pneumothorax decompression in adults. These devices are mainly catered towards military use in the treatment of pneumothorax spurred by gunshot wounds. One such device, Simplified Pneumothorax Emergency Air Release (SPEAR®), packages a needle, catheter, and one-way valve in the form factor of a pen.⁶ This device, and many others like it are accompanied by straightforward instructions and tutorials for locating the insertion point and performing the operation.

In both adults and neonates, if recurrent pneumothorax is of concern a catheter or chest tube will be inserted during the initial decompression of the lungs. This process provides practitioners with means to rapidly and repeatedly expel air from the pleural cavity without additional tissue puncturing. Both chest tubes and catheters remain in the patient's body until full resolution of the pneumothorax is observed. While the treatment of pneumothorax in adults is well defined with various commercially available products, the training process and means of decompression for neonates is less defined due to the sensitivity and relatively small market for such products.

The aim of neonatal pneumothorax decompression is to alleviate built-up air pockets inside the chest wall that threaten the functionality of vital organs in neonates. The current procedures require a medical provider to assemble a functional decompression device using multiple modular components, which can be inefficient and time consuming in emergency situations. Additionally, two medical providers are required to issue treatment as one is needed to hold the needle and the other to manually extract air. The current procedures also use a standard butterfly needle inserted into the chest wall, which results in significant guesswork and uncertainty regarding the correct needle depth. This standard needle is not easy to stabilize during decompression and can often move causing potential harm to surrounding tissue. Thus, there is a need for a device that provides a pre-assembled design that can be quickly deployed by only one medical provider. The design must enable a single provider to insert the needle and aspirated air in one fluid motion. The device must provide an adjustable needle length with a large gripping area that can be inserted and held by the medical provider with much more positional accuracy. The device must also provide an indication when the chest cavity has been reached to avoid damaging any surrounding tissue. Finally, the decompression device must maintain a low cost which is comparable to the current method when added safety features and practicality are considered.

### BRIEF SUMMARY

A primary object of the present invention is the establishment of a functional decompression device for use in neonates that meets the crucial design requirements noted below in Table 1.

TABLE 1
Key Design Requirements

| | | Importance | | |
|---|---|---|---|---|
| | | (1 = low, | Marginal | Ideal |
| # | Requirement | 5 = high) | Value | Value |
| 1 | Number of operators | 5 | 1 | 1 |
| 2 | Biocompatible | 5 | — | — |
| | Materials | | | |
| 3 | Adjustable exposed | 5 | <1 | <1¼ |
| | needle length | | | |
| 4 | Cost for 20x units | 4 | <10 | <5 |
| 5 | Device air capacity per | 3 | >15 | 60 |
| | use | | | |
| 6 | Minimum indicator | 3 | −5 | −3 |
| | operating pressure | | | |
| 7 | Device Length | 2 | 7 | 5 |
| 8 | Device grip diameter | 3 | 2 | 1 |

Another object of the present invention is to make an alternative device that requires one operator and is safer and just as effective as commonly used devices for neonatal pneumothorax that are multi-part devices and require two skilled operators to successfully evacuate the intrapleural pressure.

Another object of the present invention is to produce a device that is single operator friendly and biocompatible.

Another object of the present invention is the establishment of a mechanism for needle length adjustment. The user of the device can adjust the length of the needle during the insertion procedure as each case is different and requires variability to account for different sized neonates. The device includes a design feature that easily changes the usable needle length and locks when the desired depth is reached.

Another object of the present invention to create a better and more robust device at a cost that is not substantially higher than the current device (e.g., under 10 USD).

Another object of the present invention is to produce a device configured to remove the correct amount of air in as few cycles as possible because this procedure is done in an emergency scenario. Based on interviews with providers performing this procedure, 15 to 60 cc of air needs to be removed to treat pneumothorax. Therefore, the device can be configured to remove 15 cc of air each cycle.

Another object of the present invention is to produce a device that is able to perform properly in the conditions of the neonate's intrapleural cavity. This object is important with respect to any pressure indicator for the device.

Other objects, features, and advantages of the present invention will be apparent to one of skill in the art from the following detailed description and figures.

### BRIEF DESCRIPTION OF THE FIGURES

The present application can be understood by reference to the following drawings, wherein like reference numerals represent like elements. The drawings are merely exemplary to illustrate certain features that may be used singularly or in combination with other features and the present application should not be limited to the embodiments shown.

FIG. 1A is an isometric side view of an embodiment of a neonatal pneumothorax decompression device in accordance with the present disclosure.

FIG. 1B is a line drawing of the neonatal pneumothorax decompression device of FIG. 1A.

FIGS. 2A-2C are isometric side views of three embodiments of a cupping pump design.

FIG. 2D is an exploded view of an embodiment of a cupping pump design.

FIG. 2E is a detailed isometric side view of an embodiment of a cupping pump and valve connection.

FIG. 2F is an isometric side view of view of an embodiment of a cupping pump with a transparent tube and graduation for precise aspiration.

FIG. 3A is an isometric side view of an embodiment of a spinner airflow indicator.

FIG. 3B is an isometric side view of an embodiment of a ball airflow indicator.

FIG. 4 is an isometric side view of an embodiment of a stabilizer ring at the distal end of a needle stabilizer.

FIG. 5A is an exploded view of an embodiment of a ratcheting mechanism for adjusting needle length.

FIG. 5B is an isometric side view of the ratcheting mechanism of FIG. 5A in the device housing.

FIG. 6 is a photograph of an embodiment of a functional prototype of a neonatal pneumothorax decompression device.

FIG. 7 is an isometric side view of an embodiment of a pump cylinder with dimensions as indicated.

FIGS. 8A and 8B are photographs of an embodiment of a model for determining pump volume of a neonatal pneumothorax decompression device.

FIG. 9 is an isometric side view of an embodiment of a clear pump cylinder with volumetric labels.

FIG. 10 is a photograph of an embodiment of a model for determining pump force of a neonatal pneumothorax decompression device.

FIGS. 11A and 11B are photographs of an embodiment of a model for testing leakage from a neonatal pneumothorax decompression device.

FIGS. 12A-12D are line drawings of various embodiments of a pressure calibrated one-way valve device for use in decompressing a tension pneumothorax while allowing for chest tube placement.

### DETAILED DESCRIPTION

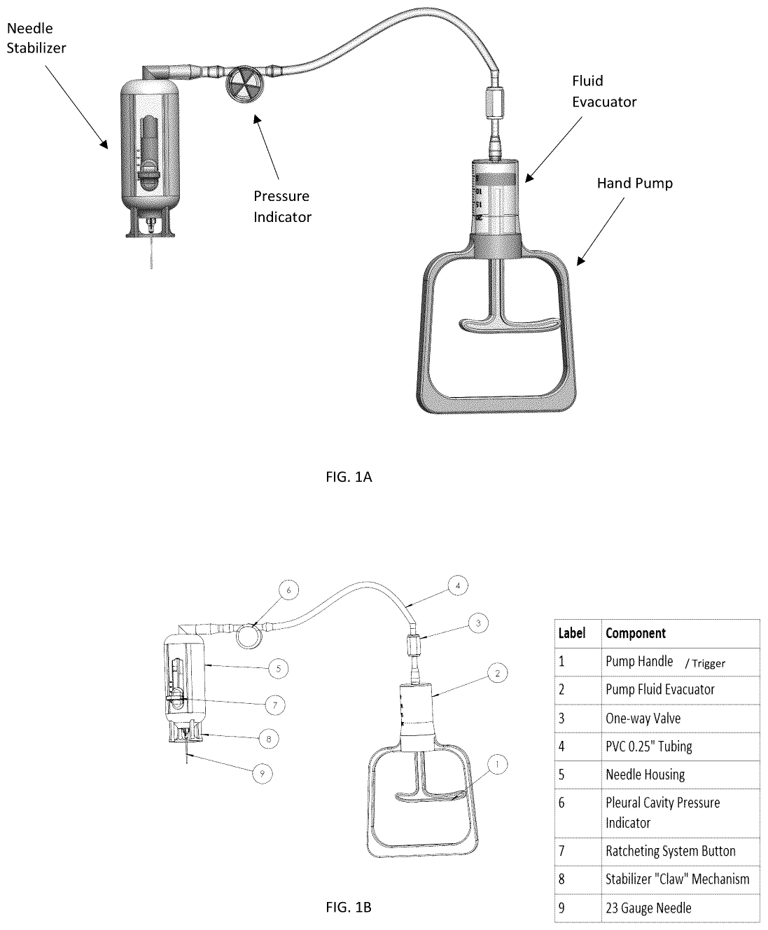

Referring now to the drawings wherein like reference numerals are used to identify like elements in the various views, FIG. 1A illustrates a device specifically designed to treat neonates for pneumothorax using decompression. The design was created based on the emergency nature of the procedure and has accessibility and ease of use as the basis of the design. To describe the device, it can be broken down into several components: the hand pump, the fluid evacuator (as used herein, the term “fluid” includes both liquids and gases), the pressure indicator, and the needle stabilizer. The components can be connected by a ¼″ inner diameter medical-grade PVC tubing attached to ¼″ plastic barb fittings.

The full device and its components are shown in FIG. 1B. The pump can be actuated using one hand due to the location of the pump handle and trigger (hand pump) 1. The pump fluid evacuator 2 is attached to a one-way valve 3 so the air aspirated from the pleural cavity is released to the open air and does not re-enter the body. In an embodiment, the indicator 6 is a bright red device that spins once negative pressure is reached. This signals the user that the pleural cavity has been reached and the user can begin aspiration.

The stabilizer 8 serves two primary design requirements. It helps the user stabilize the device on the patient's chest during needle insertion and while aspiration is performed. The needle housing 5, which can be a part of or attached proximally to the stabilizer 8, includes a ratcheting mechanism 7 that controls the height of the needle; the button on the housing can move 2 mm increments of needle length at a time so the user has complete control during needle insertion. This also assists in assuring that the needle does not reach a depth that can puncture vital organs such as the lungs.

The three components are combined with medical grade tubing 4 with the hand pump 1 on one end and the indicator 6, needle 9 and stabilizer 8 on the other end as shown in FIG. 1B. The user can follow these general steps to use the device:
1. The second intercostal space is located just above the third rib and the patient's skin is cleaned.
2. The stabilizer is placed on the chest with the second intercostal space in the center of the stabilizer.
3. The needle is inserted by pushing the button on the needle housing to release 2 mm of the needle at a time.
4. The user stops actuating the needle after the red indicator begins spinning. The pleural cavity has been reached.
5. The user may begin aspirating air by pulling back on the handle of the hand pump. This step is repeated as necessary until the pneumothorax is eliminated.

### Fluid Pumping Device

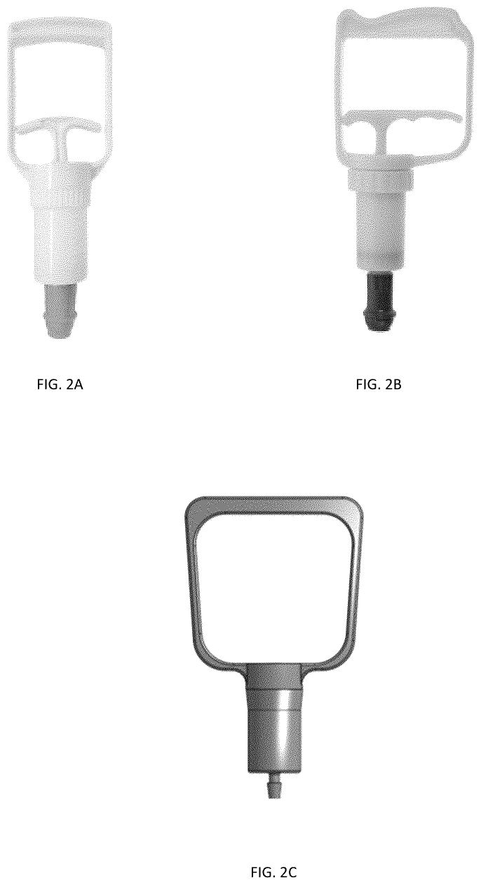

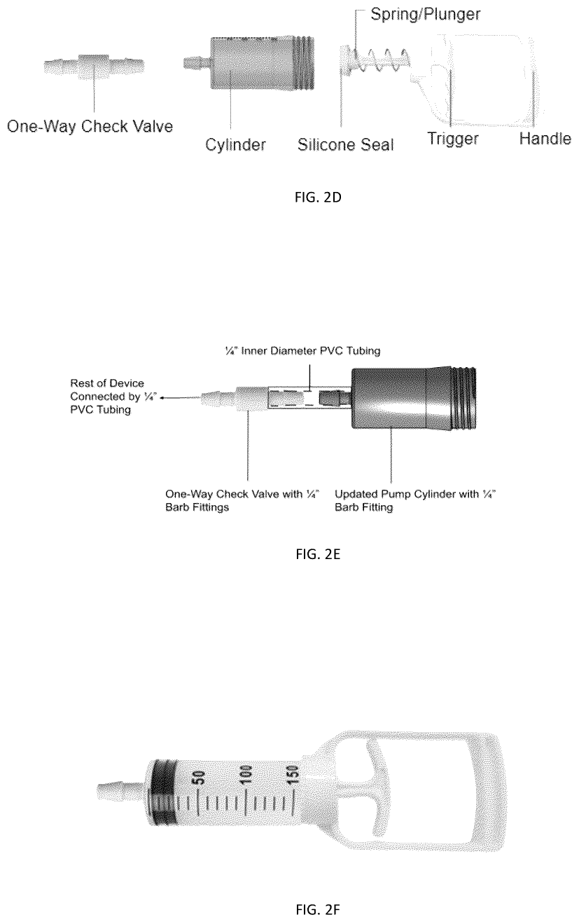

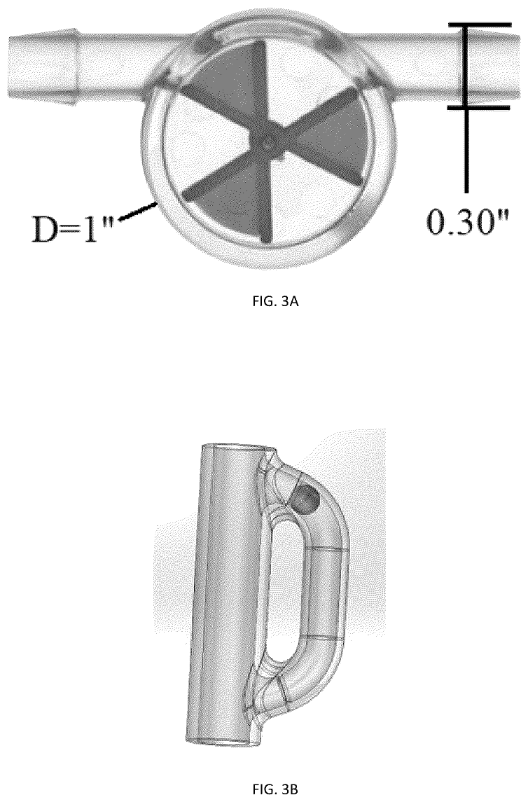

The first main component of the neonatal pneumothorax decompression device is the fluid pump. The fluid pump is used to aspirate air and fluid from the pleural cavity during the decompression procedure. The pump is operational with only a single hand as the other hand of the operator will be needed to hold the opposite end of the device during the procedure. In an embodiment, the pump design is based on the design of cupping therapy pumps used today in many massage therapies and holistic medicine clinics. The basic designs of these pumps can be seen in FIGS. 2A-2C.

These pump designs consist of three main parts; the handle, the pump cylinder, and the trigger/plunger assembly. These three components are consistent across most similar designs. The trigger is connected to a plunger that is spring loaded inside of the pump cylinder. Within the plunger, there is a mechanically activated silicone seal valve that pushes air within the pump cylinder out the back of the device during aspirations. This allows the user to continuously withdraw air by squeezing the trigger without having to manually empty the cylinder. The air will enter the pump once the trigger is squeezed, and will release out the back of the device once the trigger is released. The basic configuration of this device can be seen in FIG. 2D.

This device can easily incorporate a similar design for the aspiration of fluid from the pleural cavity, such as to treat pleural effusion or hemothorax. Using the same configuration, a one-way check valve can be added to the nozzle of the pump to restrict any air or fluid from going back into the patient. This allows the device to be constantly pumped with each trigger squeeze pulling air from the cavity, releasing the air out of the back of the device, all while restricting air from reentering the chest cavity.

In an embodiment, an adhesive is used to attach a one-way check valve to the nozzle of the pump. This one-way valve is glued into the ¼″ PVC tubing attached to the rest of the device. In another embodiment, the pump is designed to incorporate a barbed fitting at the end of the pump cylinder instead of the silicone nozzle. This may be more efficient as the pumping device can then be directly connected to a short section of PVC tubing. The one-way check valve is then connected to the end of that section of the PVC tubing via a barbed fitting allowing the entire pump and one-way valve assembly to be completed without any need for adhesive. The configuration of this design can be seen in FIG. 2E.

The fluid pumping device can also include volumetric labeling. The pump cylinder on can be made of a clear material and can be volumetrically labeled in cubic centimeters similarly to a graduated cylinder or a common syringe. This allows providers to visually see how much fluid is being aspirated during each trigger pull. The goal is to accommodate for a range of 15-60 cc during the procedure, so the cylinder can be designed to extract roughly 15-20 cc of fluid for one full singular cycle.

Another embodiment of the pump design is shown in FIG. 2F. This cylinder design with the integrated ¼″ barb can be constructed using commercially available pumps and barb fittings. Using this design, a silicone mold can be cast and then filled with a clear resin. The cylinder can then be appropriately labeled with the correct volumes using engraving or printed stickers.

### Indication Device

The next major component of the device is an airflow indicator that visually shows the user that the pleural cavity has been reached by the needle. Existing devices for pneumothorax decompression rely on the “feeling” of the physician who is inserting the needle to the patient. Once the pleural cavity has been reached, the slight feeling of negative pressure on the syringe plunger indicates this event to the physician. This triggering event is prone to error. Thus, the present neonatal pneumothorax decompression device includes a pressure indicator that has the ability to accurately indicate when the pleural cavity has been reached.

An embodiment of a pressure indicator is shown in FIG. 3A. This embodiment includes a spinning airflow indicator with a colored (e.g., red) component that is configured to spin as soon as airflow enters one of the barbed ends of the attached PVC tubing. The spinner is configured to be sufficiently small, such that the low pressure differential within a neonatal pleural cavity will cause the indicator to spin.

The indicator has two barbed ends that are 0.30″ at the widest point, as shown in FIG. 3A. This facilitates attachment to the ¼″ tubing for the device and ensures that there is a proper seal between the component and the tubing. The inner diameter of the indicator tubes match the PVC tubing of ¼″.

In an embodiment, the indicator is located just above the needle housing of the device. The indicator can attach to the needle housing on one end and the ¼″ PVC tubing on the other (see FIGS. 1A and 1B). The color (e.g., red) of the indicator makes it visually appealing and easy to see. Since a user will have one hand always be placed around the needle/needle housing, the placement of the indicator is designed to be in line of sight at all times. The needle housing can be 3D printed such that the indicator can easily attach to the top of it and a rubber sealing ring (such as an o-ring) may be used to ensure that it is properly sealed.

Another embodiment of a pressure indicator is shown in FIG. 3B. This embodiment includes a “ball” indicator that extends from the needle housing. The ball indicator can be brightly colored (e.g., red) so as to be easily visible. Once the pressure has changed throughout the device, the ball will move, indicating that the pleural cavity has been reached and the physician can begin aspiration. Once the pressure is equal throughout the device, the ball will return to the bottom of the tube due of gravity.

Another embodiment of a pressure indicator (not shown) includes an auditory signal, such as a whistle, to indicate airflow or a pressure differential withing the pleural cavity.

### Stabilization Device

The final piece of the device is the stabilization component, which only allows the needle's insertion depth to remain constant, but provides further stability for the operator to keep the needle from shifting and damaging tissue. Existing devices rely on the operator to stabilize the needle manually, which while effective, even a trained hand can still be subject to small shakes. Especially in the case of a distraction by some other issue or a slipping hand, the use of existing devices can allow for the needle to slip and potentially cause drastic tissue damage. The presently disclosed neonatal pneumothorax decompression device addresses this risk via the stabilization component of the device.

Using a spacer, e.g., the “claw” design, the operator can vary the depth of needle insertion, and when proper depth is reached, lock it in place. Therefore, a physical barrier can always be present for prevention of tissue or organ damage. Used in conjunction with the airflow indicator, this can also allow for more accuracy in depth control, so when the trapped air between the chest wall and lungs has been aspirated, the risk of puncturing the lungs is minimized.

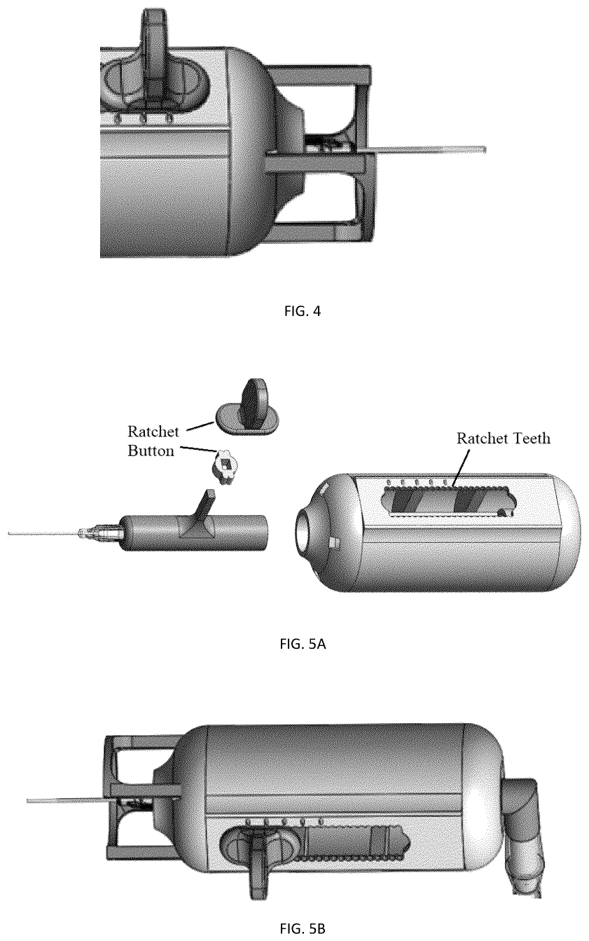

The stabilizer itself can be mounted to the device on four struts, shown in FIG. 4 as the ring that surrounds the needle. In an embodiment, the stabilizer ring is approx. 1.5″ in diameter. The ring is designed to maximize surface area (to minimize potential damage to very sensitive skin tissue) and minimize interference both with the neonate's chest and with the operator's view and dexterity. There is an opening in the ring to allow the user to place their thumb near the needle insertion location. The stabilizer is used in conjunction with the ratcheting mechanism, described below, which allows for adjustment and holding the stabilizer in place.

### Ratcheting Mechanism

As shown in FIGS. 5A and 5B, the ratcheting mechanism allows the user to adjust the exposed needle length from 0 to ¾″ in increments of 2 mm. ¾ inches is the maximum depth physicians have described in order to reach the intrapleural space and aspirate air. In an embodiment, the tip of the needle can be completely sheathed. In an embodiment, the increment distance can be 1-2 mm.

In an embodiment, the ratcheting mechanism that moves the needle is a compliant mechanism actuated by a button that moves the needle along the vertical axis of the device. When the button is pressed, the user can push the plastic button through a small interference ratchet, and can move the needle up or down. When the button is released, the needle will stay in place and cannot move more than the 2 mm ratchet distance. The material of the ratchet teeth is polyactic acid (PLA) and the material of the ratchet button is nylon, so that the button is softer and easier to move past the PLA teeth.

The ratcheting device provides the user with quantitative feedback on the needle length. It also gives very precise control and is easy to actuate. Additionally, the ratchet mechanism allows the needle to be fully retracted into the device, increasing the safety of the device when not in use or ready for disposal. Since the device may be disposable and only be used once, the degradation of the ratchet teeth caused by friction is not a concern.

### Use

Use of the neonatal decompression device can be broken down into three crucial steps including identifying and cleaning the insertion location, stabilizing and inserting the needle, and finally pumping fluid out of the pleural cavity. Prior to use of the decompression device, an ultrasound can be used to confirm the presence of a pneumothorax and identify if there is fluid contained within the cavity. If any liquid is observed, a standard Chest Tube procedure is used. If the cavity contains air, the simplified decompression device may be used. The neonate is laid in the supine position and given oxygen. The needle insertion location is determined. Depending on the ultrasound results, the needle is either be inserted in just above the rib in the second intercostal space or in the in the fifth intercostal space along the anterior axillary line. The selected site will then be cleaned and prepped for needle insertion.

Once an insertion location has been established, the stabilization device is be pressed firmly against the chest wall via the user's dominant hand. The practitioner's non-dominant hand is used to press the button located on the stabilization device, thus lowering the needle into the chest cavity. Upon reaching the intrapleural cavity, the indication wheel will begin to spin due to a pressure gradient between the chest cavity and pressure within the decompression device. Once this is observed, the needle has reached an acceptable depth and can safely be stabilized using a single hand.

The user's non-dominant hand is then used to actuate the pumping mechanism and expel gas from within the chest wall. The practitioner continues pumping and taking note of the total air expelled until the indication device is no longer spinning, indicating the pressure within the chest cavity and decompression device is at equilibrium. The user then releases the pump and uses both hands to carefully withdraw the needle from the chest cavity, ensuring the needle remains normal to the chest wall. The insertion location is treated, and the neonate's vitals continue to be monitored. The device can then be separated at the indicator tube joint, allowing the stabilizer to be properly disposed of in a sharps waste. The pump may then be disposed of however the user sees fit.

### Tension Pneumothorax Decompression Device Allowing for Chest Tube Placement

Another embodiment of the present invention is a device configured to evaluate air/fluid from the pleural cavity of a patient with a tension pneumothorax while also allowing for subsequent chest tube placement. This device can be used in neonates, children, and adults.

A tension pneumothorax develops when there is increased pressure inside the pleural cavity that exceeds atmospheric pressure. This pressure causes tension physiology which decreases cardiac output and can lead to hypotension and cardiac arrest.

Standard of care treatment of patients with a tension pneumothorax involves needle decompression either in the midclavicular line, second intercostal space, or mid-axillary line 4^{th}-5^{th} intercostal space, followed by chest tube insertion for more definitive treatment. This is important because the decompression of the tension pneumothorax does not treat the underlying pathology which created the pneumothorax. Chest tube insertion is often needed after needle decompression to provide supportive therapy to the pleural space allowing the underlying lung insult/injury that caused the pneumothorax time to heal. Chest tube insertion after tension pneumothorax decompression carries its own set of risks, however, because once the air/fluid has been evacuated from the intrapleural space the lung is more likely to have expanded to contact the pleural lining and the risk of lung puncture during chest tube insertion increases.

Methods have been devised to equalize the pressure between a tension pneumothorax and atmospheric pressure using a decompression needle. See, e.g., U.S. Pat. No. 9,616,203B2, which is herein incorporated by reference in its entirety. This method allows the user to treat a tension pneumothorax while providing confirmation of pressure differences between the atmosphere and intrapleural cavity. The disadvantage of the valve, however, is that there is no guard against the pleura re-adhering to the chest wall once the pressure equalizes.

The present invention overcomes deficiencies in the prior art by maintaining some pressurized air/fluid to remain in the intrapleural space to allow room for chest tube insertion. A primary object of the invention is to create a pressure valve which decompresses the tension pneumothorax without completely evacuating the intrapleural cavity. This involves releasing pressure in the intrapleural cavity that would cause tension physiology to develop while simultaneously maintaining a small amount of positive pressure (e.g., +5 to +10 cm H₂O) above atmospheric pressure in the intrapleural cavity to provide more space within the pleural cavity to insert a chest tube. When left in place, the decompression needle with attached valve can continue to operate and allow excess buildup of tension physiology level pressure air/fluid to be evacuated from the intrapleural space until the chest tube is inserted.

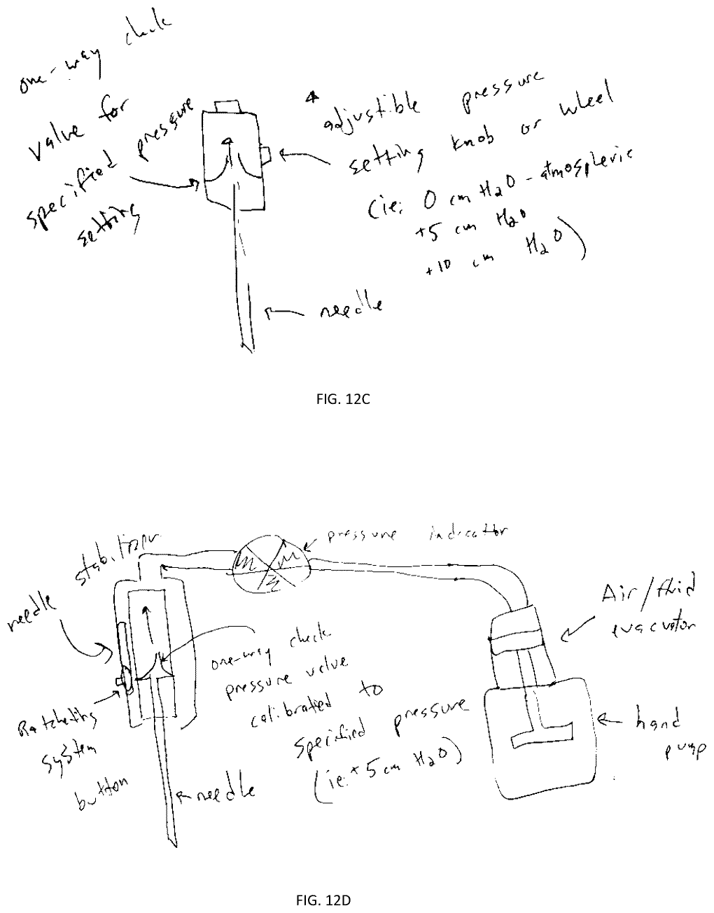

As shown in FIG. 12A, a one-way valve calibrated to a specified pressure (e.g., +5 to +10 cm H₂O) can be incorporated into a needle housing (e.g., needle housing 5 shown and described above with respect to FIG. 1B). The needle housing can include a tubing connector (e.g., for use with medical grade tubing 4 shown and described above with respect to FIG. 1B), which can be used to connect the device to a pressure indicator (e.g., indicator 6 shown and described above with respect to FIG. 1B), hand pump (e.g., pump handle and trigger 1 shown and described above with respect to FIG. 1B), syringe aspirator, or other device component. The one-way valve is configured to let air/fluid escape the pleural cavity, but not enter it. The one-way valve comprises material which is calibrated to allow air/fluid over a predefined pressure setting to escape the pleural cavity, and to allow air/fluid under the predefined pressure to remain in the pleural cavity. The one-way valve device is configured to remain in place within the pleural space. When the pressure in the pleura exceeds the predefined pressure setting of the device, air/fluid will be evacuated until the pressure reaches the predefined pressure setting of the device. When the pressure in the pleura reaches the predefined pressure setting, the valve will close and air/fluid movement will stop.

FIG. 12B illustrates an embodiment, similar to that of FIG. 12A, in which the pressure indicator is incorporated into the needle housing of the one-way valve device.

FIG. 12C illustrates an embodiment, similar to that of FIG. 12A, including a pressure setting knob or wheel coupled to the needle housing of the one-way valve device. The pressure setting knob or wheel can be tightened or loosened to adjust the pressure setting of the one-way valve.

FIG. 12D illustrates an embodiment in which the one-way valve device of FIG. 12A is used in conjunction with the above-described device and system, including the hand pump, fluid evacuator, pressure indicator, and needle stabilizer shown and described above with respect to FIGS. 1A-5B.

### EXAMPLES

### Example 1—Design Evaluation

### 1.1. Overview

When considering the features of the above-described neonatal pneumothorax decompression device which would need verification, most fell under qualitative analysis, and a few fell under quantitative analysis. Altogether, the evaluation included physical tests on prototypes and VOC surveys, as well as basic CAD design evaluation. Due to the minimal size and relative complexity of our device, simulations or computations would need to be greatly idealized to work, and the simplicity of 3D printing a part and testing it by hand versus creating a complicated simulation made more sense in nearly every case. We also counted on opinions of those with experience in this procedure and made many decisions based on their feedback.

TABLE 2
Design Requirements and Test Methods for Evaluation

| # | Requirement | Value | Units | Justification | Test Method |
|---|---|---|---|---|---|
| 1 | Number of operators | 1 | — | Must be single- | Test prototype on a |
| | | | | user friendly | balloon to determine |
| | | | | | ease of use for one |
| | | | | | person and measure |
| | | | | | maximum force |
| | | | | | needed to operate |
| | | | | | the pump with force |
| | | | | | gauge. |
| 2 | Exposed needle | ½-1 | in. | Works on | Use of caliper to |
| | length | | | different sized | confirm needle |
| | | | | neonates | length |
| | | | | | measurements meet |
| | | | | | specifications |
| 3 | Biocompatible | — | — | Device will be | Confirm using |
| | Materials | | | inserted into the | materials |
| | | | | body | spreadsheets that |
| | | | | | parts used in final |
| | | | | | design would be |
| | | | | | biocompatible |
| 4 | Able to remove air | — | — | Device must be | Volumetric testing |
| | from chest cavity | | | able to remove | of air capacity for |
| | | | | air within chest | the cylinder in the |
| | | | | cavity to treat | pump using a |
| | | | | pneumothorax | balloon inflated |
| | | | | | with a known |
| | | | | | volume. |
| 5 | Device must be | — | — | Stabilization | The prototype was |
| | stable after insertion | | | component will | given to users and |
| | | | | prevent over- | tested using a |
| | | | | insertion and | surgical infant |
| | | | | tissue damage | mannequin |
| 6 | Device indicates | — | — | User will need to | The selected |
| | when the chest cavity | | | know when to | indicator was |
| | has been reached. | | | stop insertion | presented to |
| | | | | and begin | practitioners and |
| | | | | aspiration | visibility was |
| | | | | | recorded |
| 7 | Grip diameter | ¾-2 | in. | Appropriately | The diameter of the |
| | | | | sized for better | prototype stabilizer |
| | | | | control of the | grip was measured |
| | | | | device | |
| 8 | Device air capacity | 10-60 | cc | Evacuate air in a | Volumetric testing |
| | | | | minimal # of | of pump design |
| | | | | cycles | using known |
| | | | | | volumes |
| 9 | Minimum operating | −15-15 | cmH20 | The device must | The indicator and |
| | pressure | | | be able to | pump were able to |
| | | | | function inside | properly function |
| | | | | the chest cavity | under the standard |
| | | | | | balloon training |
| | | | | | procedure |
| 10 | Overall device length | 3-7 | in | Appropriately | The total length of |
| | | | | sized for | the prototype |
| | | | | emergency use + | stabilizer was |
| | | | | convenience | measured |

### 1.2. Prototype

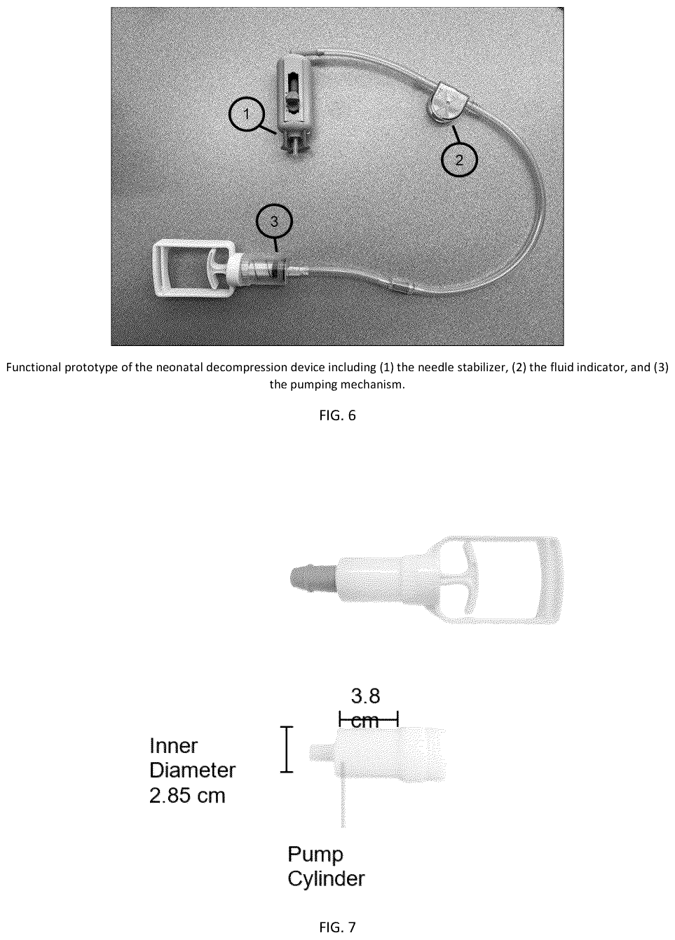

A functional prototype for the neonatal decompression device was established to allow practitioners to give valuable feedback on design criteria and make iterative changes based on responses. The final prototype, as seen in FIG. 6 allows for testing of the overall functionality and ergonomics of the device. This prototype is split into the three main components of the decompression device, including (1) the needle stabilizer, (2) the indication device, and (3) the pumping mechanism. The needle stabilizer allows the user to stabilize the needle with a single hand while allowing for incremental insertion into the chest wall. Several secondary goals of this device include fully concealing the needle during storage, modularity to allow for sharps disposal, and allowance for various needle insertion techniques that practitioners use. The indication device consists of a commercially available spinning indicator, allowing the user to visibly see when air is flowing. The final aspect of this device includes the pumping mechanism, which serves to ensure the device can be operated by a single user and can evacuate the volume of the pneumothorax with one or more pump cycles. When all three components of the design are combined the needle can be inserted into a pressurized chamber and air may be evacuated via the air pump by a single operator.

### 1.3. Testing and Results

### 1.3.1. Geometric Analysis

### Introduction

The decompression device must adhere to strict geometric constraints in order to remain functional, practical, and ergonomic. The overall length of the stabilizer is crucial to establishing a device compatible with current NICU procedures of needle aspiration (described in Background Section). Additionally, the diameter of the stabilizer grip ensures that the device is not cumbersome and can be operated by a single practitioner. Finally, the maximum length of the needle confirms that the proposed device will function over a wide variety in neonate age and size. These three design requirements have been lumped into a single geometric analysis as they are experimentally measured using identical methods. While these constraints were met or exceeded during the design phase, this section will serve to confirm these results via measurements of a physical prototype and note any discrepancies between the design and prototype.

### Methods

The design requirements previously described were all tested on the prototype by measuring the length and diameter of the stabilizer as well as the fully extended length of the needle. The only required equipment for these tests was a ruler, caliper, or similar measurement device. In measuring the length of the stabilization device, measurement was taken from the base of the support, where the device contacts the skin, to the top of the adapter elbow, where the stabilizer is then connected via tubing to the indication device. The diameter of the stabilizer was measured at the point of largest cross-sectional area around the device. The maximum length of the needle was measured by fully extending the needle past the stabilization device and measuring the distance from the tip of the needle to the base of the stabilizer. Each measurement was taken once.

### Results and Discussion

The total length of the stabilization device was measured to be 4.61 inches from skin contact to tube connection. This can be compared to the digital model with a total length of 4.6 inches, indicating slightly decreased resolution in the chosen fabrication method of standard FDM 3D printing. This added length is undesirable, but the prototype still complies to the design criteria for a maximum length of 3-7 inches. It should be noted that the device length is highly dependent on the length of the hypodermic needle being used. In this case, a standard 23 G needle with a length of 1 inch was selected. By minimizing the total length of the device within the acceptable range the cost of bulk material and manufacturing time can be reduced.

The diameter of the device was measured to be 1.61 inches. This value is again slightly greater than the expected diameter of 1.6 inches based on the designed components due to resolution limits in 3D printing. This value falls within the upper limit of the design requirement range of ¾-2 inches. It was selected to increase the diameter of the device based on various interviews with users which are detailed in subsequent analyses.

Finally, the maximum length of the needle was measured to be 0.98 inches. This value falls within the specified range for maximum needle length of ½-1 inch. It should be noted that the device designed is capable of reaching various depths within this range of 0.1 inch increments. The prototype presented was created with relatively low resolution FDM printing in order to meet the expected budget. It can be seen that a tradeoff exists between manufacturing resolution and component cost. With each geometric constraint met or exceeded in the low cost prototype, it can reasonably be stated that the decompression device will reach the depth range essential for evacuating air from the pleural cavity in a wide variety of neonatal morphologies and is appropriately sized to sit at the NICU bedside for immediate use if a pneumothorax develops.

### 1.3.2. Functionality Analysis

### Introduction

Developing a functional decompression device that is preferred by practitioners is the goal of both design and analysis. In order to meet these goals several design requirements must be met. The entire device must be single-user operated and include a component for stability of the needle during the procedure. Additionally, the device must be able to properly evacuate air from the chest cavity and indicate that air is being released. The analysis conducted to meet these constraints includes interviews and surveys with 18 total practitioners over three separate occasions. This allowed for iterative design based on the needs and preferences of users with diverse medical backgrounds.

### Methods

To determine if the device can be operated by a single user, various prototypes were presented to practitioners who were then asked to operate the device without supplying them with any additional training. In doing so, an unbiased viewpoint of whether the device was single-user friendly or not was achieved. This process was conducted for the different prototypes, ensuring that each subsequent iteration continued to meet this design criteria. The stability of the decompression device was not measured numerically as this is highly variable based on neonate age, condition, and size. It was opted to test the stability of the device based solely on ergonomics and visibility of the needle as it is being inserted. Various alternatives for stabilizers were proposed to practitioners and their feedback was recorded to be used in further iterations. In addition to providing feedback on stabilization, users were also given the opportunity to comment on the design as a whole, indicating their preferred forms of indication and pumping as well as any additional aspects they would like included.

The operating pressure range, ability to remove air, and indicator actuation were observed and assessed via the creation of a low-cost training device to be paired with the decompression device. This training device consists of an inflated balloon with a piece of tape over one section to prevent the balloon from popping upon needle insertion. By inflating a standard balloon, similar conditions to the pleural cavity experiencing pneumothorax can be achieved. The needle will then be lowered into the balloon and the indicator will be viewed during insertion. Once secured to the balloon, the user will then begin pumping air out until the balloon has been fully deflated, indicating that all three previously described design requirements have been met.

### Results and Discussion

Various alternatives for stabilization and indication were first presented to practitioners of various backgrounds. Through survey results it was found that every interviewee preferred a device with a ratcheting stabilizer and a spinning indicator. These choices were made over other alternatives including a friction-fit stabilizer and ball or heimlich valves for indication. Each prototype was then presented and it was observed how each practitioner expected to use the device. Ultimately, each practitioner was able to simulate the procedure without assistance indicating the device is single user-friendly. It should be noted that some differences in procedure existed based on dominant hand and user background. It was observed that left-handed users would hold the device from the side, while right-handed users held the device from the top. This difference stemmed from the placement of the connecting tube atop the stabilizer, which was then modified to allow usage to be independent of hand preference. Additionally, NICU nurses desired an opening to allow room for the thumb during needle insertion while pediatric surgeons were impartial to this design aspect. The preference of NICU nurses was weighted greater than that of pediatricians and pediatric surgeons as these nurses were the predominant users of this device. With this in mind, an opening for the thumb was then added to the stabilizer. A full list of additional recommendations and comments can be found in Appendix L (incorporated herein by reference as part of U.S. Provisional Application Nos. 63/453,617 and 63/606,285) as the voice of the customer.

The functionality of the final device was then compared against a low-cost training apparatus. It was observed that upon needle insertion the indicator began to spin briefly, indicating that the balloon, or cavity had been reached. The pump was then actuated at various speeds until all air was evacuated from the balloon. It was observed that the indicator continues to spin over the course of evacuation and once the balloon has been fully deflated the spinner begins to slow. This trial ultimately does not confirm whether the design requirements have been met as there is considerable discrepancy between a balloon and the standard neonatal intrapleural cavity. It should be noted that the balloon method is common practice when training new practitioners, indicating its resemblance to the actual procedure on an infant. Under the assumption that these current training procedures provide similar pressures to real neonatal pneumothorax decompression, it can reasonably be assumed that the proposed device will function as intended upon proper sterilization and packaging.

### 1.3.3. Volume Analysis

### Introduction

One design requirement focuses on the device air capacity, specifically the pump air capacity. The required air capacity falls within the range of 10-60 cubic centimeters for any single procedure. Due to the variable size of patients and their respective chest cavities, this is a necessary volume requirement to cover any neonate that needs the procedure. Since the air evacuator is a spring activated hand pump, the volume of evacuated air needs to be measurable and easily visible to the provider during the procedure. The approach to the design of the pump was to mimic a standard clear syringe with printed volumetric markings as shown in the design concept in FIG. 2F. The overall goal of this test is to verify the pump can extract a range of 10 cc-60 cc and that the volumetric markings printed on the pump are providing accurate feedback. It is assumed that the pump is completely sealed (no air leakage) and there is no air stored within the lines or cylinder beforehand.

### Methods

The goal of this test is to verify the pump is properly aspirating a range of 10-60 cubic centimeters and that the markings printed on the pump are accurately measuring the necessary extracted volume during each aspiration.

The volume of the pump can be theoretically calculated using the length and diameter of the clear cylinder section of the pump. The dimensions of the pump and the calculations for theoretical volume can be shown in FIG. 7. Using the dimensions shown in FIG. 7, the theoretical pump cylinder volume can be calculated as:

$$3.8 cm \times {π ( 2.85 cm / 2 )}^{2} = 24.24 cubic centimeters \qquad ( 1 )$$

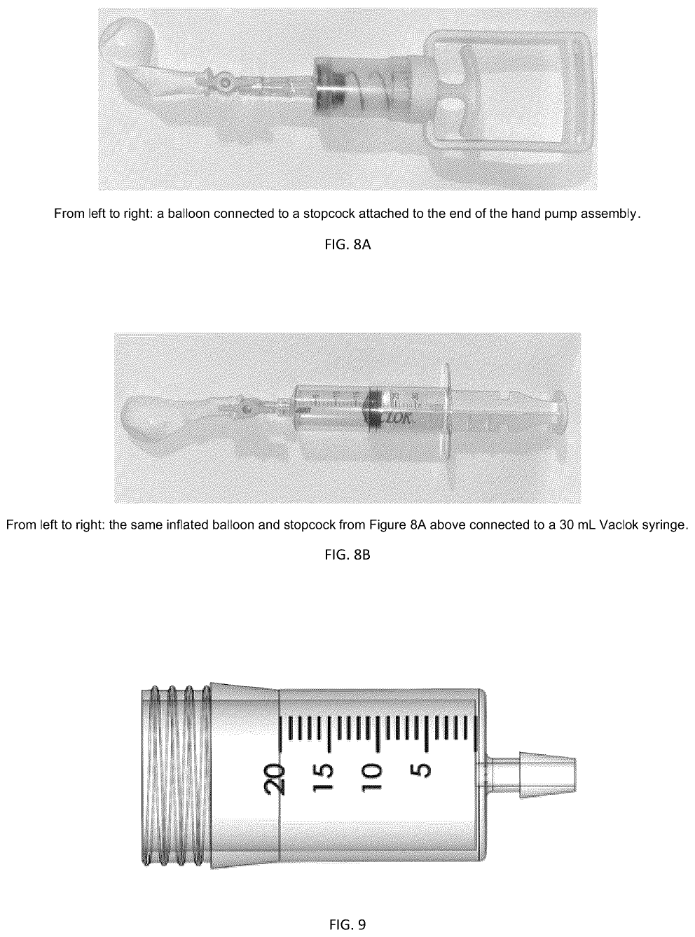

The volume of the pump can be experimentally determined by extracting an unknown amount of air from a balloon until it is empty using a labeled volumetric syringe with a known volume. In this experiment, the hand pump will be fully retracted and connected to a balloon and stopcock within the inline assembly as shown in FIG. 8A.

The hand pump will then be released and the air inside the pump cylinder will enter the balloon. The stop cock will be shut and the pump will be replaced with a 30 mL volumetrically labeled syringe as shown in FIG. 8B.

The contents of the balloon will then be removed by opening the stopcock and pulling vacuum on the volumetric syringe. Once the balloon is empty, the stopcock will be closed and the amount of fluid can be measured by looking at the volumetric displacement of the plunger within the syringe. Five trials will be conducted using this process and the final volumetric displacements of each trial will be averaged to calculate the experimental air capacity of the hand pump cylinder.

Once the air capacity of the cylinder in the hand pump is known, the volumetric markings can then be accurately labeled on the cylinder by dividing the known volume from the amount of desired markings. These markings can then be printed equally spaced along the outside of the clear pump.

### Results and Discussion

TABLE 3
Volume of air extracted from balloon using a 30
mL volumetric syringe for five hand-pump trials.

| Trial | Final Volume Extracted From Balloon | |
|---|---|---|
| 1 | 21 | mL |
| 2 | 20 | mL |
| 3 | 19 | mL |
| 4 | 22 | mL |
| 5 | 20 | mL |
| Average | 20.4 | mL |

After five trials, the average volume of air extracted from the balloon was calculated to be 20.4 mL. This gives the hand pump an experimental air capacity of 20.4 cc.

This experimental value is roughly 4 cc less than the theoretical air capacity calculated above. This can be due to the connections between the tubing and the pump not being completely sealed during the experiment. It can also be due to leftover air within the balloon after retracting air using the syringe as the two components may not have created a perfect vacuum during the measuring process. Another source of error could come from the dimensions of the pump not having been uniform across the whole part as the cylinder was re-molded in silicone. This could mean that the diameter of the cylinder may not be exactly 2.85 cm throughout the full length which could decrease the expected air capacity. Overall, the experimental and theoretical values were close while taking into account the possible sources of error.

Using the experimental air capacity of 20.4 cc, the cylinder of the pump can be divided into 4 equal sections of roughly 5 cc and labeled along its length for the purpose of this prototype. In future models, the volumetric labeling would be much more precise than this initial prototype as it could be etched into the cylinder mold or printed using an automated stamp during post processing.

The overall clear pump design with the proper volumetric labeling on the cylinder can be seen below in FIG. 9.

### 1.3.4. Pump Force Analysis

### Introduction

A key design requirement for this product is the ability for a single user to operate the device during the entire procedure. The justification of this design requirement is that the product can be marketed as single-user friendly, reducing the amount of time and people needed to perform the procedure with this new device. In order to quantify “single user friendly,” the force needed to operate the pump can be explored; as this parameter must be low enough that the operator can aspirate the device with one hand continuously. Since this is determined to be the most difficult part of the procedure to do with only one person, this will be the only parameter tested for the characterization of “single-user friendly.”

### Methods

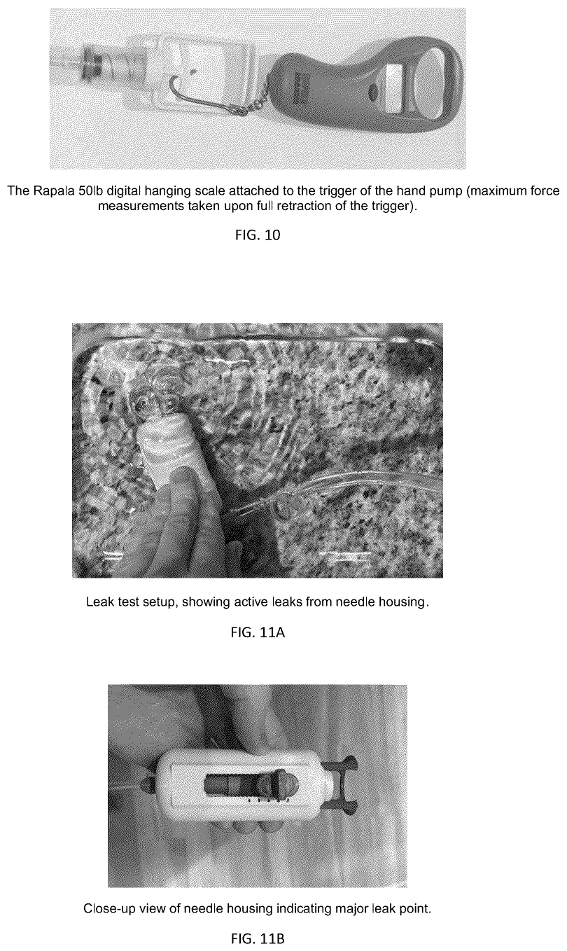

The force needed to fully retract the aspiration pump can be measured using a handheld force gauge or similarly with a handheld electronic hanging scale. In this analysis, a Rapala 50 lb handheld electronic hanging scale will be attached to the trigger of the aspiration pump as shown below in the experimental setup within FIG. 10.

Over 10 trials, the pump will be fully retracted while attached to the end of the electronic hanging scale. The maximum force will be recorded and averaged over the 10 trials. This data can then be compared with a variety of average grip strength by age within multiple data sheets found in related studies. The comparison of these numerical values will then determine if the pump can efficiently be operated with one hand allowing the overall device to be characterized as “single-user friendly.”

### Results and Discussion

TABLE 4
Maximum force measured upon full trigger
retraction over ten trials.

| Trial | Maximum Force Measured |
|---|---|
| 1 | 7.23 lb |
| 2 | 7.80 lb |
| 3 | 7.89 lb |
| 4 | 7.73 lb |
| 5 | 8.13 lb |
| 6 | 7.40 lb |
| 7 | 7.35 lb |
| 8 | 7.91 lb |
| 9 | 7.71 lb |
| 10 | 7.87 lb |
| Average | 7.69 lb |

The maximum force was measured 10 separate times in various grip positions within the pump trigger. As shown in Table 4 above. The average maximum force measured throughout all ten trials was calculated to be 7.69 pounds.

In order to characterize the device as “single-user friendly,” the maximum pump actuation force must be significantly less than the average single-handed grip strength for the age range of 40 for a typical neonatal nurse. This will show that the pump can be comfortably actuated for an extended period of time with one hand which allows the nurse to efficiently use their other hand for holding the stabilizer.

A range of grip strengths by age for men and women for a repeated grip motion between 10 and 15 times is can be seen below in pounds.¹⁰
Ages 35-39: Right 95.7-143.7; Left 91.2-134.6
Ages 40-44: Right 96.1-137.5; Left 94.1-131.5
Ages 45-49: Right 86.9-132.9; Left 78-123.6

The data for ages between 35-49 are shown above. Since the average neonatal nurse age is 40 and because this value is subject to change, the wider range above was used for comparison. The lowest value within this data group is a grip strength of roughly 78 pounds. This value is nearly 10× larger than the average maximum force for the pump actuation measured at 7.69 pounds.

Using this data from the ten trials and the known grip strength data for the applicable age range, the device can be successfully characterized as “single-user friendly.” It is reasonable for the average nurse to be expected to actuate 7.69 pounds of force a maximum of three times during the procedure. This can all be done with the ability to hold the stabilizer in the other hand.

### 1.3.5. Leak Test

### Introduction

Lining up with the device's requirements to aspirate air effectively and in a measurable manner, the device's air aspiration channel should be free of leaks. If the device cannot maintain air capacity at small pressures, its readings will not be held as accurate and there can be no assurance that ambient air isn't being added to the measurement of aspirated air.

### Methods

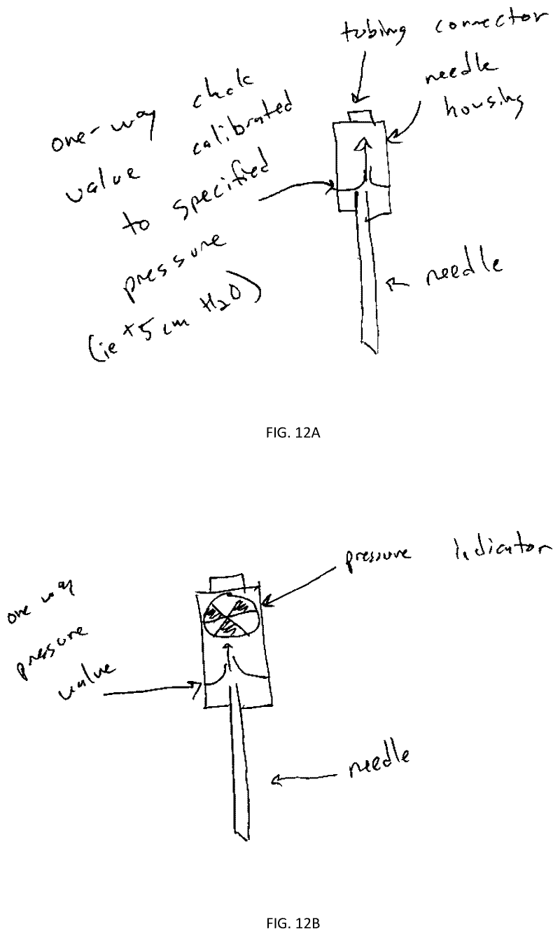

The goal of this test is to verify the pump can maintain an air-tight seal at operating pressures. Therefore, we will qualitatively analyze if leaks occur by sealing the needle end with a luer stopper fitting, applying a small amount of pressure with the syringe, and lowering the entire needle housing into a vat of water. If bubbles appear at any fitting area or connection point, we know that the given area is susceptible to leaks. The setup is shown in FIGS. 11A and 11B.

This test assumes that manufactured luer lock fittings are leak proof, at least in the scope of this device, which is a completely reasonable conclusion.

If it is deemed possible to easily fix without fundamental design alterations, the proper step will be taken and the leak test repeated until no leaks remain, and the failure points will be recorded for evaluation and prevention in a manufacturing capacity. Through this test, we can verify that our device will not be giving improper readings or losing air, and we can come out of it with a good understanding of potential failure points in a production device.

### Results and Discussion

The device clearly leaks from inside the main housing, meaning all barb and luer fittings can be trusted, but the internal seal of the needle slider is likely very leak-prone, as a lot of bubbles are coming from that region. It's possible the PLA is leaking slightly as well, but this is more likely negligible compared to the O-ring seal. It doesn't make sense to try to find a quick fix for this problem because it's likely a fitment and tolerancing issue from the O-ring. This would and should take some serious analysis and consideration (nearly a full redesign of this component) to properly fix in a way that keeps the smooth operation of the ratcheting needle system.

The obvious and most detrimental leak point is the O-ring seal in the slider, which makes sense from a design perspective. This is the largest surface area of sealing in the device, which means it would need a huge amount of sealing force from a mechanical seal in order to stand up to moderate pressures. In a full redesign of this component, it would be sensible to decrease the diameter of the slider to limit the surface area of sealing, as well as finding a tighter-toleranced O-ring for the sake of getting a better mechanical seal.

### 1.4. Assessment

The prototype developed served its purpose well and can be used to assess if this design should be implemented in practice. The device only requires a single operator to use; it has an adjustable needle length to work on different sized neonates and allow for safe disposal; it has a stabilizer mechanism to avoid inserting the needle too far; and removes the proper amount of air. All of these features were mentioned as primary customer needs, and the prototype meets all of those needs. Additionally, the materials used were stainless steel, PLA, and nylon, all of which are used in medical applications and safe for short term implants. The prototype also has multiple safety and usability benefits, including but not limited to, the indicator, needle stabilizer, and the needle being fully encapsulated within the housing of the ratcheting components. This can be compared to the current needle aspiration method which lacks all of these features. Finally, the prototype has received positive feedback and interest in implementation from roughly 15 practitioners. The prototype appears to meet all primary customer needs; and the interviewed users of this device are interested in using this product.

The prototype developed has a few shortcomings that need to be addressed. First, there is no sterilization method for this prototype. A sterilization cycle and sterilized pouches would need to be designed before the device can be used in practice. Second, the device cannot remove any undesired fluid in the body, meaning that another device must be on hand in hospitals in case this situation arises. Third, a few components used were stock components. This allows ease of use, but cost could be lowered if the components were designed and manufactured in house. Finally, parts are designed for 3D printing which is good for prototyping but the design would need to be reworked if large scale manufacturing processes such as injection molding will be used.

Further details regarding the disclosed neonatal pneumothorax decompression device prototype can be found in Appendices A-M, which are incorporated herein by reference as part of U.S. Provisional Application Nos. 63/453,617 and 63/606,285.

Although at least one embodiment of a neonatal pneumothorax decompression device has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The terms “about” and “approximately” may be used throughout the specification when referring to a measurable value, such as an amount, a distance, a temporal duration, and the like. The terms “about” and “approximately” are meant to encompass variations of 20% or ±10%, in certain embodiments ±5%, in certain embodiments ±1%, in certain embodiments ±0.1% from the specified value, as such variations are appropriate in accordance with the present disclosure.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

### REFERENCES

1. Choi, Won-Il. “Pneumothorax.” Tuberculosis and Respiratory Diseases, vol. 76, no. 3, 29 Mar. 2014, p. 99., doi.org/10.4046/trd.2014.76.3.99.
2. Nelson, Nicholas M. “Neonatal Pulmonary Function.” Pediatric Clinics of North America, vol. 13, no. 3, 1966, pp. 769-799, doi.org/10.1016/s0031-3955(16)31882-x.
3. Dinwiddie, R, and G Russell. “Relationship of Intraesophageal Pressure to Intrapleural Pressure in the Newborn.” Journal of Applied Physiology, vol. 33, no. 4, 1972, pp. 415-417, doi.org/10.1152/jappl.1972.33.4.415.
4. “Curosurf® (Poractant Alfa): Official HCP Website.” Curosurf, 18 Jan. 2022, curosurf.com.
5. Vibede, Louise, et al. “Neonatal Pneumothorax: A Descriptive Regional Danish Study.” Neonatology, vol. 111, no. 4, 2016, pp. 303-308, doi.org/10.1159/000453029.
6. “Spear—Simplified Pneumothorax Emergency Air Release.” Simplified Emergency Air Release|North American Rescue.
7. Pocket Book of Hospital Care for Children: Guidelines for the Management of Common Illnesses. World Health Organization, 2013.
8. Hadzic, Devleta, et al. “Risk Factors and Outcome of Neonatal Pneumothorax in Tuzla Canton.” Materia Socio Medica, vol. 31, no. 1, March 2019, p. 66, doi.org/10.5455/msm.2019.31.66-70.
9. Esme, H., Dogru, O., Eren, S., Korkmaz, M., & Solak, O. (2008). The factors affecting persistent pneumothorax and mortality in neonatal pneumothorax. The Turkish Journal of Pediatrics, 50(3), 242.
10. “Hand Grip Strength Test” Livestrong Fitness (2019) www.livestrong.com/article/468905-hand-grip-strength-test/

What is claimed is:

1. A device for treating pneumothorax in a subject, the device comprising:
a needle stabilizer comprising a proximal end and a distal end, the needle stabilizer configured to hold a needle at an adjustable distance from the distal end and to facilitate insertion of the needle into the subject's pleural cavity;
a pressure indicator coupled to the proximal end of the needle stabilizer, the pressure indicator configured to visibly display pressure within the subject's pleural cavity; and
a fluid evacuation pump configured to be actuated by a user to evacuate fluid from the subject's pleural cavity through the needle, wherein the fluid evacuation pump is proximally coupled to the pressure indicator and the needle stabilizer.

2. The device of claim 1, wherein the subject is a neonate.

3. The device of claim 1, wherein the device is biocompatible.

4. The device of claim 1, wherein the device is configured to be operated by a single user.

5. The device of claim 1, wherein the needle stabilizer, the pressure indicator, and the fluid evacuation pump are coupled via medical tubing.

6. The device of claim 1, wherein the fluid evacuation pump comprises:
a handle;
a pump cylinder; and
a trigger/plunger assembly comprising a one-way valve configured to expel aspirated air or fluid from a proximal end of the device.

7. The device of claim 6, wherein the trigger/plunger assembly further comprises a trigger attached to a plunger that is spring-loaded inside the pump cylinder.

8. The device of claim 6, wherein the trigger/plunger assembly is configured to be actuated by the user with one hand.

9. The device of claim 6, wherein the pump cylinder is configured to hold about 15-60 cc of aspirated air or fluid.

10. The device of claim 1, wherein the fluid evacuation pump is configured to aspirate about 15-20 cc of air or fluid per pump cycle.

11. The device of claim 1, wherein the pressure indicator comprises an airflow spinner configured to spin when the needle enters the subject's pleural cavity and encounters a pressure gradient.

12. The device of claim 11, wherein the airflow spinner is configured to stop spinning when pressure within the subject's pleural cavity and pressure within the device is at equilibrium.

13. The device of claim 1, wherein the needle stabilizer comprises a ratcheting mechanism configured to adjust a length of the needle exposed at the distal end of the needle stabilizer by a fixed increment distance.

14. The device of claim 13, wherein the length of the needle exposed at the distal end of the needle stabilizer is from about 0 to about ¾ inches, and wherein the fixed increment distance is about 1 to about 2 mm.

15. The device of claim 13, wherein the ratcheting mechanism includes a button configured to be actuated by the user, and wherein actuation of the button moves the needle along a vertical axis of the needle stabilizer.

16. The device of claim 13, wherein the ratcheting mechanism is further configured to lock the needle in place when the needle has reached the subject's pleural cavity.

17. The device of claim 1, wherein the needle stabilizer further comprises a needle housing and a stabilizer ring mounted to a distal end of the needle housing, and wherein the needle stabilizer is configured to extend the needle distally through a central opening of the stabilizer ring.

18. The device of claim 17, wherein the stabilizer ring is mounted to needle housing via a plurality of struts.

19. A method of using the device of claim 1 to treat pneumothorax, the method comprising:
a) identifying an insertion location on the subject's chest;
b) placing the needle stabilizer on the insertion location;
c) inserting and lowering the needle into the subject's pleural cavity until the pressure indicator signals a pressure gradient;
d) pumping the fluid evacuation pump to aspirate gas or fluid from the subject's pleural cavity until the pressure indicator stops signaling a pressure gradient;
e) withdrawing the needle from the subject's pleural cavity.

20. The method of claim 19, further comprising using ultrasound to confirm the insertion location based on a location of the pneumothorax prior to using the device.
